(19) United States
(12) Patent Application Publication
(10) Pub. No.: US 2008/0139573 A1
(43) Pub. Date: Jun. 12, 2008

(54) Treatment of resistant Schizophrenia and other CNS disorders

(75) Inventors: Jean-Michel Azorin, Marseille (FR); Christophe Lancon, Marseille (FR)

Correspondence Address: DICKSTEIN SHAPIRO LLP, 1177 AVENUE OF THE AMERICAS (6TH AVENUE), NEW YORK, NY, 10036-2714, US

(73) Assignee: Copharm, Luxembourg (LU)

(21) Appl. No.: 11/635,677

(22) Filed: Dec. 8, 2006

(51) Int. Cl. A61K 31/497; A61K 31/382; A61P 25/18

(52) U.S. Cl. 514/252.13; 514/437

(57) ABSTRACT

A method of mono-therapeutically treating a patient suffering from a condition selected from naïve schizophrenia, therapy resistant schizophrenia, therapy refractory schizophrenia, therapy resistant depression, chronic depression, recurrent depression, and resistant bipolar disorder comprises the administration of a pharmacologically effective amount of a thioxanthene, such as chlorprothixene, flupenthixol, thiotixene, zuclopenthixol, zuclopenthixol acetate and zuclopenthixol decanoate, including a pharmaceutically acceptable salt thereof. Also disclosed is a corresponding use and the manufacture of a corresponding medicament.

### FIELD OF THE INVENTION

The present invention relates to a method of treating resistant depression, chronic and recurrent depression and resistant bipolar disorders. In particular the invention relates to the treatment of nave schizophrenic patients, therapy resistant schizophrenic patients, and therapy refractory schizophrenic patients. The invention also relates to a means for use in the method.

### BACKGROUND OF THE INVENTION

Schizophrenia is one of the most debilitating diseases that psychiatrists have to treat. Schizophrenia is a psychiatric condition that is characterised by delusion, hallucination, disorganized speech, grossly disorganised or catatonic behaviour and negative symptoms (i.e. affective flattening, alogia, or avolition). Schizophrenia is a severe condition associated with increased mortality (two to three times the average for the general population), comorbidity as well as social exclusion.

Life time prevalence is high, one percent, and may vary between different studies depending on the definition criteria of the target population. It is also known that the prevalence increases with age until the age of 40 where after it declines. The course of illness is characterized by relapses which occur in 80% of individuals over a two year period. The disease starts in men in their late teens or early 20s and for women in their 20s or early 30s. According to NIMH, schizophrenia affects men and women with equal frequency.

In the United States, it is estimated that about 2.4 million individuals suffer from schizophrenia, out of which 20% to 30% are patients with little or no response to antipsychotics and normally referred to as therapy-resistant patients. This means that 480,000 to 720.000 schizophrenic patients are left with no efficient therapy. This is a highly vulnerable population exposed to a significant suffering.

The pharmacological management of schizophrenia is based on typical antipsychotics (haloperidol, chlorpromazine, thioridazine, fluphenazine, perphenazine, trifluoroperazine, amisulpride, sulpiride,) which induce motor side effects and extrapyramidal side effects (EPS). The development of a new generation of antipsychotics called atypicals, such as risperidone, olanzapine, quetiapine, ziprasidone and aripiprazole, was initially perceived as a breakthrough in the management of schizophrenia as they were thought to be void of EPS while at the same time offering better efficacy. The new atypicals were thought to be efficacious enough to address the need of the 20-30% of schizophrenic patients therapy resistant to typical antipsychotics. However, a recent study concluded that atypical antipsychotics are not more efficacious than typicals. The evidence of superior efficacy of atypical antipsychotics over typical was neither consistent nor robust and the safety advantage of atypical versus typical has been questioned because of their ability to induce weight gain and altering glucose and lipid metabolism and disturbing cardiac repolarisation.

The only antipsychotic agent that is indicated in therapy resistant schizophrenic patients is clozapine. However, its use is limited due to severe side effects. Clozapine's potential side effects include a loss of disease-fighting white blood cells with neutropenia in 3.6% and agranulocytosis in 1%, an overall seizure rate of 2.8%, and potentially myocarditis with resultant cardiomyopathy and fatal heart failure and pulmonary embolism. Despite it's potentially lethal side effects, clozapine was approved by the US FDA for a restricted population: therapy resistant schizophrenia. Careful periodic monitoring of blood cell count is mandatory to diagnose the occurrence of agranulocytosis and therefore strongly limits its use.

When antipsychotic treatment is initiated, 75% of the patients will discontinue treatment within 18 months due to inappropriate response such as insufficient relief of symptoms or treatment related side effects.

In this application therapy resistant schizophrenic patient, therapy refractory schizophrenic patient and naïve schizophrenic patient is defined as follows.

Therapy resistant schizophrenic patient is a schizophrenic patient who has tried two successive antipsychotic agents for a period of 6 weeks at an appropriate dosage with no significant symptomatic relief of symptoms and who was compliant to pharmacological treatment. This definition comprises determining relief of symptoms by measurement on a clinical scale such as PANSS (positive and negative schizophrenia scale). The relief should correspond to a reduction of the PANSS scale of at least 20% after a period of 6 weeks treatment of an appropriate dosage. Lack of relief of symptoms should be assessed prospectively and should not be documented on the basis of historical information. Compliance to treatment should be documented through a third party such as nurse or care giver. Lack of compliance is often a reason for unsatisfactory outcome of schizophrenia therapy; if compliance is not documented the diagnosis of therapy resistant schizophrenia cannot be ascertained. Appropriate dosage is defined as an equivalent chlorpromazine of 400-700 mg per day.

Therapy refractory schizophrenic patient is a schizophrenic patient who, after 6 weeks of treatment with an antipsychotic agent, has achieved significant relief of symptoms, but who nevertheless exhibits residual symptoms that prevent the patient to achieve remission.

Naïve schizophrenic patient is a schizophrenic patient who had never been treated with an antipsychotic agent.

In a naïve schizophrenic patient:

The course of illness of schizophrenia is characterized by more or less frequent relapse defined as an acute exacerbation of schizophrenic symptoms: the higher the number of previous relapses, the higher risk of further relapse. In a naïve patient, early control of symptoms with longer relapse-free periods patient is predictive of a better outcome. A treatment that would delay occurrence of relapse in naïve patient would fulfil an important unmet need and would dramatically affect the long term course of schizophrenia.

The object of the present invention is to provide a pharmacological treatment that delays the occurrence of relapse thus addressing an important unmet medical need in naïve patients

Therapy resistant and therapy refractory schizophrenic patients: clozapine is normally used for these patients but, as has been described above, it is linked to serious side effects thus limiting its use. Apart from clozapine, some schizophrenia unrelated drugs have been used as adjuvant in treatment-resistant schizophrenic patients such as:
famotidine (N′-(aminosulfonyl)-3-[[[2-[(diaminomethylene)amino]-4thiazolyl]methyl]thio]propanimidamide) (J Psychiatry Neurosci 1994; 19(2): 145-150)
D-serine (Am J Psychiatry 1999, 156:1822-1825)
galantamine ((4aS,6R,8aS)-4a,5,9,10,11,12-hexahydro-3-methoxy-11-methyl-6H-benzofuro[3a,3,2-ef](2) benzazepin-6-ol) (Am J Psychiatry 2002 159(7): 1244-5

Some other compounds have also been used for treatment-resistant schizophrenia when used as monotherapy:
melperone (4-fluoro-γ-(4-methylpiperidino)-butyrophenone) U.S. Pat. No. 5,221,679 (1993)
amperozide (N-Ethyl-4-(4′,4′-bis(4-fluorophenyl)butyl)-1-piperazinecarboxamide) U.S. Pat. No. 5,013,735 (1991)

However, none of those agent are approved by a regulatory agency, such as FDA in the USA, for the treatment of schizophrenia to date.

Although pharmacotherapeutic approaches, such as clozapine, seem to play a role in the management of therapy resistant and therapy refractory schizophrenic patients, there is still a strong unmet need in this population. In particular, there is a need for an alternative pharmacological option with a more favorable safety profile than clozapine.

Recurrent, chronic and resistant depression and resistant bipolar disorders

It has been estimated that about 20-30% of patients with major depression fail to respond to treatment with a single antidepressant drug given in adequate dosage for an appropriate period. In addition, only about half such patients will respond when switched to another antidepressant medication.

Since lack of full response is common, management strategies need to be established and implemented. Current approaches include: the use of combination antidepressant treatments (e.g., selective serotonin reuptake inhibitor and bupropion), augmentation (e.g., the addition of lithium or thyroid hormone to an antidepressant), and the addition of psychotherapy or electroconvulsive therapy (Shelton, 2003, 1999). However, even with these approaches, a significant number of patients do not experience a full therapeutic effect.

We hereafter define the terms: recurrent depressive patient, chronic depressive patient, resistant depressive patient and resistant bipolar disorder patient:

Recurrent depressive patient is defined as a patient experiencing two or more successive major depressive episodes that are separated by at least two months of not experiencing adequate symptoms of depression to qualify for major depressive disorder (DSM-IV).

Chronic depression patient is defined as a patient experiencing two years or more of persistent depressive symptoms (DSM-IV).

Resistant depression patient is defined as a patient not responding to an appropriate pharmacological treatment.

Resistant bipolar disorder patient is defined as a patient not responding to an appropriate pharmacological treatment.

Recurrent depression, chronic depression, resistant depression, and resistant bipolar disorder are correspondingly defined.

The object of the present invention is to provide a pharmacological treatment that addresses the unmet medical need in therapy resistant and therapy refractory schizophrenic patients by providing an efficacious pharmacological option with a more favourable safety profile than clozapine. Another objective of the present invention is to provide a pharmacological treatment that offers an early control of symptoms and delays occurrence of relapse in naïve schizophrenic patients.

It is also the object of the present invention to provide a pharmacological treatment that addresses the unmet medical need in recurrent depressive patients, chronic depressive patients, resistant depressive patients and those suffering from resistant bipolar disorders.

### SUMMARY OF THE INVENTION

It has unexpectedly been found that thioxantenes, such as thiotixene, chlorprothixene, flupenthixol, zuclopenthixol, zuclopenthixol acetate, zuclopenthixol decanoate, pharmaceutically acceptable salts, prodrugs and mixtures thereof,
are effective as mono-therapy in therapy resistant schizophrenic patient
are effective as monotherapy in therapy refractory schizophrenic patient
delay occurrence of relapse in naïve schizophrenic patient when used as monotherapy.

In particular, it has unexpectedly been found that zuclopenthixol is an efficient drug for therapy resistant schizophrenic patients not responding to drugs such as haloperidol, risperidone, olanzapine and clozapine and that zuclopenthixol is an efficient drug for naïve schizophrenic patient where it delays relapse

On the other hand, it has also been found that thioxantenes such as thiotixene, chlorprothixene, flupenthixol, zuclopenthixol, zuclopenthixol acetate, zuclopenthixol decanoate, pharmaceutically acceptable salts, prodrugs and mixtures thereof, are effective as mono-therapy in
patients with resistant depression
patients with recurrent depression
patients with chronic depression
patients suffering from resistant bipolar disorders.

According to the present invention is disclosed a method of treating a patient suffering from a condition selected from naïve schizophrenia, therapy resistant schizophrenia, therapy refractory schizophrenia, therapy resistant depression, chronic depression, recurrent depression, and resistant bipolar disorder, comprising the administration of a pharmacologically effective amount of a thioxanthene of the general formula (I) as monotherapy, although they can also be used in combination therapy if desired.

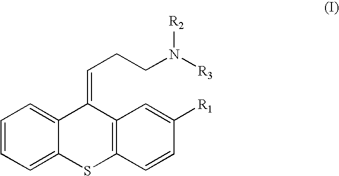

(Ia) R₁=Cl, R₂=R₃=CH₃
(Ib) R₁=CF₃, with NR₂R₃=4-(2-hydroxyethyl)-1-piperazinyl
(Ic) R₁=SO₂(NCH₃)₂, with NR₂R₃=4-methyl-1-piperazinyl
(Id) R₁=Cl, with NR₂R₃=4-(2-hydroxyethyl)-1-piperazinyl
(Ie) R₁=Cl, with NR₂R₃=4-(2-acetyloxyethyl)-1-piperazinyl
(If) R₁=Cl, with NR₂R₃=4-(2-decanyloxyethyl)-1-piperazinyl

Specific thioxanthenes include, but are not limited to chlorprothixene (Ia; (3Z)-N,N-dimethyl-3-(2-chloro-9H-thioxanthen-9-ylidene)propan-1-amine), flupenthixol (Ib; 4-[(3Z)-3-(2-trifluoromethyl-9H-thioxanthen-9-ylidene)propyl]-1-piperazineethanol), thiotixene (Ic; (3Z)-N,N-dimethyl-9-[3-(4-methyl-1-piperazinyl)-1-propylidene]-9H-thioxanthene-2-sulfonamide), zuclopenthixol (Id; 4-[(3Z)-3-(2-chloro-9H-thioxanthen-9-ylidene)propyl]-1-piperazineethanol dihydrochloride), and esters of compound (Id) such as (Ie; zuclopenthixol acetate) and (If; zuclopenthixol decanoate), and pharmaceutically acceptable salts thereof.

### DETAILED DESCRIPTION OF THE INVENTION

The present invention provides a method of treating naïve schizophrenic patients, therapy resistant schizophrenic patients and therapy refractory schizophrenic patients by orally administering a thioxanthene, preferably selected from the group consisting of thiotixene, chlorprothixene, flupenthixol, zuclopenthixol, zuclopenthixol acetate, and zuclopenthixol decanoate. In a preferred embodiment the invention provides a method of treating naïve schizophrenic patients, therapy resistant schizophrenic patients and therapy refractory schizophrenic patients by oral administration of zuclopenthixol.

Administration for gastrointestinal absorption can be by the oral or rectal route, such as by tablets and suppositories, respectively. It is also within the scope of the invention to provide liquid formulations, in particular aqueous formulations, for oral administration. A further route of administration is by intravenous or intramuscular injection in a liquid carrier such as physiological saline. Pharmaceutical compositions of the compounds of the invention suitable in the treatment of naïve schizophrenic patients, therapy resistant schizophrenic patients, therapy refractory schizophrenic patients, and patients suffering from resistant depression, chronic and recurrent depression and resistant bipolar disorders are described in literature and are marketed, albeit for different indications, in various countries.

A pharmacologically effective oral dose of zuclopenthixol dose from 5 mg to 400 mg, preferably from 20 mg to 150 mg, is given as monotherapy to naïve schizophrenic patients, therapy resistant schizophrenic patients, and therapy refractory schizophrenic patients.

When given in such a dose, zuclopenthixol exerts a clinically beneficial effect on symptoms of schizophrenia, in particular on positive symptoms, and on cognitive dysfunction. No deterioration of any other aspect of schizophrenia, such as cognitive or negative symptoms or excitement, accompanies the beneficial effects of zuclopenthixol on positive symptoms, even during long term administration of zuclopenthixol, such as over more than half a year and even a year or more.

The therapeutical efficiency of the compounds of the invention in regard of the conditions to which it relates is supported by the following examples.

### EXAMPLE 1

### Therapy Resistant Schizophrenia

Case 1. A therapy resistant schizophrenic patient (37y) did not respond to the successive administration of four antipsychotic agents, each prescribed for a sufficient period of time and at a relevant dosage, and for whom compliance was controlled. After having been successively given oral haloperidol, oral risperidone, oral olanzapine, and oral clozapine, at appropriate doses and for sufficient duration, she was put exclusively on oral zuclopenthixol. Compliance to treatment was ascertained. Before being given zuclopenthixol, the patient presented severe debilitating schizophrenic symptoms. Soon after the start of zuclopenthixol treatment, the patient experienced a major relief of schizophrenic symptoms allowing her to return to normal life.

Case 2. Female subject (59 y) diagnosed with therapy resistant schizophrenia for the first time at the age of 40. The patient had been treated with oral amilsulpride, oral olanzapine, oral risperidone, and oral clozapine at appropriate doses and for sufficient duration. Compliance with treatment was ascertained. The patient was then switched to oral zuclopenthixol. Before initiating zuclopenthixol treatment, the patient was considered as “markedly ill”. After 8 weeks of treatment with zuclopenthixol, the patient was evaluated as having “very much improved”, as confirmed by C.G.I. and P.A.N.S.S.

Case 3. Male subject (29 y) diagnosed with therapy resistant schizophrenia for the first time at the age of 20. The patient had been treated with appropriate doses and for sufficient duration with oral olanzapine, oral risperidone, oral loxapine, and oral clozapine. Compliance with treatment was ascertained. The patient was then switched to oral zuclopenthixol. Before initiating zuclopenthixol treatment, the patient was considered as “among the most extremely ill patients”. After 8 weeks of treatment with zuclopenthixol, the patient was evaluated as having “much improved”, as confirmed by C.G.I. and P.A.N.S.S.

Case 4. Male subject (46 y) diagnosed with therapy resistant schizophrenia for the first time at the age of 25. The patient had been treated with appropriate doses and for sufficient duration with the following antipsychotics: oral risperidone, oral aripiprazole, oral loxapine, and oral olanzapine. Compliance with treatment was ascertained. The patient was then switched to oral zuclopenthixol. Before initiating zuclopenthixol treatment, the patient was considered as “severely ill”. After 8 weeks of treatment with zuclopenthixol, the patient was evaluated as having “much improved”, as confirmed by C.G.I. and P.A.N.S.S.

Case 5. Male subject (30 y) diagnosed with therapy resistant schizophrenia for the first time at the age of 20. The patient had been treated with oral clozapine, oral risperidone, aripiprazole, and oral olanzapine. The patient was then switched to oral zuclopenthixol. Before initiating zuclopenthixol treatment, the patient was considered as “severely ill”. After 8 weeks of treatment with zuclopenthixol, the patient was evaluated as having “much improved”, as confirmed by C.G.I. and P.A.N.S.S.

### EXAMPLE 2

### Therapy Refractory Schizophrenia

Female subject (27 y) diagnosed with therapy refractory schizophrenia for the first time at the age of 22 The patient had been treated with appropriate doses and for sufficient duration with oral aripiprazole, oral loxapine and oral olanzapine at appropriate doses and for sufficient duration. Compliance with treatment was ascertained. The patient exhibited significant relief of symptoms, but was nevertheless suffering from significant troublesome residual symptoms. The patient was then switched to oral zuclopenthixol. After 8 weeks of treatment with zuclopenthixol, the patient was evaluated as having “much improved”, as confirmed by C.G.I. and P.A.N.S.S., allowing the patient to achieve remission.

### EXAMPLE 3

### Naïve Schizophrenia

Upon diagnosis of schizophrenia, a naïve schizophrenic patient (19y) was given zuclopenthixol 50 mg p. o. per day as his first antipsychotic treatment, which proved to be successful. After a period of 5 years of treatment, the patient had still not relapsed.

### EXAMPLE 4

### Recurrent Depression

Female subject (48 y) diagnosed with recurrent depression for the first time at the age of 19. The patient had been treated with appropriate doses and for sufficient duration with oral fluoxetine, oral citalopram, oral amytriptiline and oral paroxetine but was regularly experiencing new episodes of depression. Compliance with treatment was ascertained. She was then switched to oral zuclopenthixol and exhibited a marked response. After 2 years, the patient had still not relapsed.

### EXAMPLE 5

### Therapy Resistant Depression

Male subject (33 y) diagnosed with chronic depression for the first time at the age of 23. The patient had been treated with appropriate doses and for sufficient duration with oral sertraline, oral paroxetine and oral amytriptiline without satisfactory response. He was then switched to oral zuclopenthixol. Before initiating oral zuclopenthixol treatment, the patient was considered as “markedly ill”. After 8 weeks of treatment with oral zuclopenthixol, the patient was evaluated as having “much improved”, as confirmed by C.G.I. and exhibited a marked response by reduction of depression on the Hamilton Depression Scale.

### EXAMPLE 6

### Therapy Resistant Bipolar Disorder

Case 1. Male subject (39 y) diagnosed with therapy resistant bipolar disorder I presented as having manic state. He had been diagnosed for the first time at the age of 22. The patient had been treated with appropriate doses and for sufficient duration with oral lithium and oral lamotrigine without satisfactory response. Compliance with treatment was ascertained. He was then prescribed oral zuclopenthixol. Before initiating oral zuclopenthixol treatment, the patient was considered as “among the most extremely ill patients”. After 8 weeks of treatment with zuclopenthixol, the patient was evaluated as having “much improved”.

Case 2. Female subject (43 y) diagnosed with therapy resistant bipolar disorder 11 presented with depressive symptoms. She had been diagnosed for the first time at the age of 18. The patient had been treated with appropriate doses and for sufficient duration with oral lithium, oral sertraline and oral citalopram without satisfactory response. Compliance with treatment was ascertained. She was then prescribed oral zuclopenthixol. Before initiating oral zuclopenthixol treatment, the patient was considered as “markedly ill”. After 8 weeks of treatment with zuclopenthixol, the patient was evaluated as having “very much improved”.

1. A method of treating a patient suffering from a condition selected from naïve schizophrenia, therapy resistant schizophrenia, therapy refractory schizophrenia, therapy resistant depression, chronic depression, recurrent depression, and resistant bipolar disorder, comprising administering a pharmacologically effective amount of a thioxanthene or a pharmaceutically acceptable salt, prodrug or mixture thereof.

2. The method of claim 1, wherein the thioxanthene is of the general formula (I) 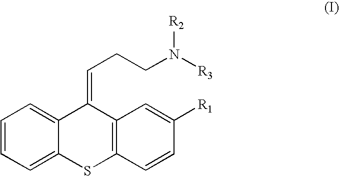
wherein
(Ia) R₁=Cl, R₂=R₃=CH₃
(Ib) R₁=CF₃, with NR₂R₃=4-(2-hydroxyethyl)-1-piperazinyl
(Ic) R₁=SO₂(NCH₃)₂, with NR₂R₃=4-methyl-1-piperazinyl
(Id) R₁=Cl, with NR₂R₃=4-(2-hydroxyethyl)-1-piperazinyl
(Ie) R₁=Cl, with NR₂R₃=4-(2-acetyloxyethyl)-1-piperazinyl or
(If) R₁=Cl, with NR₂R₃=4-(2-decanyloxyethyl)-1-piperazinyl.

3. The method of claim 1, wherein the thioxanthene is chlorprothixene (Ia).

4. The method of claim 1, wherein the thioxanthene is flupenthixol (Ib).

5. The method of claim 1, wherein the thioxanthene is thiotixene (IC).

6. The method of claim 1, wherein the thioxanthene is zuclopenthixol (Id).

7. The method of claim 5, wherein the thioxanthene is an ester of zuclopenthixol selected from zuclopenthixol acetate (1e) and zuclopenthixol decanoate (1f).

8. The method of claim 1, wherein the thioxanthene is administered in a pharmaceutical composition for per-oral administration.

9. The method of claim 1, wherein the thioxanthene is administered in a pharmaceutical composition for rectal administration.

10. The method of claim 1, wherein the thioxanthene is administered in a pharmaceutical composition for intravenous or intramuscular administration.

11. The method of claim 1, wherein said condition is therapy resistant schizophrenia.

12. The method of claim 10, wherein said condition was resistant to treatment by one or more antipsychotic agents selected from the group consisting of chlorpromazine, haloperidol, olanzapine, quietapine, risperidone, ziprasidone, aripiprazole.

13. The method of claim 1, wherein said condition is schizophrenia in a therapy refractory schizophrenic patient.

14. The method of claim 1, wherein said condition is schizophrenia in a naive schizophrenic patient.

15. The method of claim 1, wherein said pharmacologically effective amount corresponds to from 5 mg to 400 mg of zuclopenthixol daily per os.

16. The method of claim 14, wherein said pharmacologically effective amount corresponds to from 20 mg to 150 mg of zuclopenthixol daily per os.

17. A method of treating a patient suffering from a condition selected from naïve schizophrenia, therapy resistant schizophrenia, therapy refractory schizophrenia, therapy resistant depression, chronic depression, recurrent depression, and resistant bipolar disorder, comprising administering from 5 mg to 400 mg of zuclopenthixol or a pharmaceutically acceptable salt, prodrug or mixture thereof, daily per os.

18. The method of claim 15, wherein the amount administered is 20 mg to 150 mg.
